(19) United States
(12) Patent Application Publication
(10) Pub. No.: US 2025/0206107 A1
(43) Pub. Date: Jun. 26, 2025

(54) FRAGRANCE UNIT AND FRAGRANCE ASSEMBLY

(71) Applicant: Ford Global Technologies, LLC, Dearborn, MI (US)

(72) Inventors: Shuhan Ma, Nanjing (CN); Wei Xu, Nanjing (CN); Zhenhai Kong, Nanjing (CN); Xu Zhengdong, Nanjing (CN)

(73) Assignee: Ford Global Technologies, LLC, Dearborn, MI (US)

(21) Appl. No.: 18/953,329

(22) Filed: Nov. 20, 2024

(30) Foreign Application Priority Data
Dec. 21, 2023 (CN) 202323509908.9

(51) Int. Cl. B60H 3/00; A61L 9/12

(52) U.S. Cl. CPC B60H 3/0007; A61L 9/12; A61L 2209/133

(57) ABSTRACT

A fragrance unit and fragrance assembly for a vehicle having a fragrance box with a bottom wall and a side wall, a fragrance medium located inside the fragrance box, and a fluid channel extending along a first direction of the fragrance box. The fluid channel has an inlet and an outlet, with one of the inlet and the outlet located on the bottom wall and the other on the side wall, to allow airflow to flow into the fluid channel from the inlet, pass through the fragrance medium, and then exit the fluid channel from the outlet. The fluid channel is arranged around the fragrance medium.

### CROSS-REFERENCE TO RELATED APPLICATION

This application claims foreign priority benefits under 35 U.S.C. § 119 (a)-(d) to China Application No. 202323509908.9 filed Dec. 21, 2023, which is hereby incorporated by reference in its entirety.

### FIELD OF THE DISCLOSURE

The present disclosure generally relates to motor vehicles, and more specifically relates to a fragrance unit and a fragrance assembly for use on a motor vehicle.

### BACKGROUND OF THE DISCLOSURE

Fragrance distribution devices have been proposed for use in a motor vehicle. For example, a fragrance distribution device may be configured to distribute fragrance from a fragrance medium to air in the vehicle. The fragrance distribution device generally may include an actuator having a mechanism configured to actuate a valve device for each fragrance containing box between a sealed state for blocking passage of air through a fragrance medium of the fragrance containing box and an unsealed state for allowing air to pass through the passage of the fragrance medium of the fragrance containing box towards an outlet. It would be desirable to provide for enhanced fragrance distribution in a vehicle.

### SUMMARY OF THE DISCLOSURE

According to a first aspect of the present disclosure, a fragrance unit and fragrance assembly for a vehicle having a fragrance box with a bottom wall and a side wall, a fragrance medium located inside the fragrance box, and a fluid channel extending along a first direction of the fragrance box. The fluid channel has an inlet and an outlet, with one of the inlet and the outlet located on the bottom wall and the other on the side wall, to allow airflow to flow into the fluid channel from the inlet, pass through the fragrance medium, and then exit the fluid channel from the outlet. The fluid channel is arranged around the fragrance medium.

Embodiments of the first aspect of the present disclosure can include any one or a combination of the following features:
the fragrance medium is solid that is fixed to the fragrance box through a bracket located inside the fragrance box and defines the fluid channel with the fragrance box;
multiple inlets or multiple outlets are provided;
the multiple inlets or the multiple outlets are evenly spaced around a projection of the fragrance medium on the bottom wall;
a projection of the fragrance medium on the bottom wall covers the multiple inlets or the multiple outlets;
the fragrance box has a first opening opposite to the bottom wall, and the inlet or outlet is located on the side wall, spaced apart from and adjacent to the first opening;
multiple inlets or multiple outlets are provided, and the multiple inlets or the multiple outlets are evenly arranged around the side wall;
one inlet or one outlet is provided, and the one inlet or the one outlet is circular, star-shaped, rectangular-shaped, cross-shaped or elliptical;
one inlet or one outlet is provided, and the one inlet or the one outlet is cross-shaped, circular or square-shaped; and
the fragrance unit is configured to be located on a fragrance assembly inside a vehicle.

According to a second aspect of the present disclosure, a fragrance assembly located inside a vehicle has a fragrance unit comprising a fragrance box, a fragrance medium located inside the fragrance box, and a fluid channel extending along a first direction of the fragrance box and arranged around the fragrance medium. The fluid channel has an inlet and an outlet. A carrier with an accommodation cavity is formed internally, within which the fragrance unit is located. A cover comprises a connecting portion that is connected to the carrier on one side in a sealed manner and to the fragrance box on another side.

Embodiments of the second aspect of the present disclosure can include any one or a combination of the following features:
a gap in fluid communication with the fluid channel is formed between the fragrance box and the carrier;
the gap and the fluid channel extend along the first direction;
the fragrance medium is solid that is fixed to the fragrance box through a bracket located inside the fragrance box and defines the fluid channel with the fragrance box;
multiple inlets or multiple outlets are provided;
the fragrance box has a first opening opposite to the bottom wall, and the inlet or outlet is located on the side wall, spaced apart from and adjacent to the first opening; and
one inlet or one outlet is provided, and the one inlet or the one outlet is circular, star-shaped, rectangular-shaped, cross-shaped or elliptical.

According to a third aspect of the present disclosure, a vehicle has a fragrance assembly located inside the vehicle. The fragrance assembly includes a fragrance unit comprising a fragrance box, a fragrance medium located inside the fragrance box, and a fluid channel extending along a first direction of the fragrance box and arranged around the fragrance medium. The fluid channel has an inlet and an outlet. A carrier with an accommodation cavity is formed internally, within which the fragrance unit is located. A cover comprises a connecting portion that is connected to the carrier on one side in a sealed manner and to the fragrance box on another side.

Embodiments of the third aspect of the present disclosure can include any one or a combination of the following features:
the fragrance assembly has a gap in fluid communication with the fluid channel formed between the fragrance box and the carrier; and
the gap and the fluid channel extend along the first direction.

These and other features, advantages, and objects of the present disclosure will be further understood and appreciated by those skilled in the art by reference to the following specification, claims, and appended drawings.

### BRIEF DESCRIPTION OF THE DRAWINGS

For a better understanding of the present disclosure, reference may be made to embodiments shown in the following drawings. The components in the drawings are not necessarily to scale and related elements may be omitted, or in some instances proportions may have been exaggerated, so as to emphasize and clearly illustrate the novel features described herein. In addition, system components can be variously arranged, as known in the art. Further in the figures, like reference numbers refer to like parts throughout the different figures.

FIG. 1 is a side perspective view of a motor vehicle having a fragrance assembly, according to an embodiment of the present disclosure;

FIG. 2 is a lower perspective view of a fragrance unit included in the fragrance assembly, according to one or more embodiments of the present disclosure;

FIG. 3 is an exploded view of the fragrance unit illustrated in FIG. 2;

FIG. 4 is a cross-sectional view taken through a central vertical axis of FIG. 2;

FIG. 5A is a bottom end view of a bottom wall of a fragrance box, according to a first example;

FIG. 5B is a bottom end view of a bottom wall of a fragrance box, according to a second example;

FIG. 5C is a bottom end view of a bottom wall of a fragrance box, according to a third example;

FIG. 5D is a bottom end view of a bottom wall of a fragrance box, according to a fourth example;

FIG. 5E is a bottom end view of a bottom wall of a fragrance box, according to a fifth example;

FIG. 6 is a schematic diagram of a fragrance assembly according to one or more embodiments of the present disclosure;

FIG. 7 is an exploded view of the fragrance assembly illustrated in FIG. 6;

FIG. 8 is a cross-sectional view of FIG. 6 taken along line B-B; and

FIG. 9 is an enlarged view of Section C illustrated in FIG. 6.

### DETAILED DESCRIPTION OF PREFERRED EMBODIMENTS

Embodiments of the present disclosure are described below. However, it should be understood that the disclosed embodiments are only examples, and other embodiments may take various and alternative forms. Drawings are not necessarily drawn to scale; and some functions may be exaggerated or minimized to show details of specific components. Therefore, the specific structural and functional details disclosed herein should not be interpreted as restrictive, but merely as a representative basis for teaching those skilled in the art to use the present disclosure in various ways. As will be understood by those of ordinary skill in the art, various features shown and described with reference to any one of the drawings may be combined with features shown in one or more other drawings to produce embodiments which is not explicitly shown or described. The combination of the features shown provides a representative embodiment for a typical application. However, various combinations and modifications of features consistent with the teachings of the present disclosure may be expected for certain specific applications or embodiments.

In this application document, when an element or part is referred to as being “on . . . ”, “bonded to”, “connected to”, or “coupled to” another element or part, the element or part can be directly on the another element or part, can be bonded, connected or coupled to the another element or part, or there may be intervening elements or parts. In contrast, when an element is referred to as being “directly on . . . ”, “directly bonded to”, “directly connected to”, or “directly coupled to” the another element or part, the intervening elements or parts may not be present. Other words used to describe the relationship between elements should be interpreted in a like fashion.

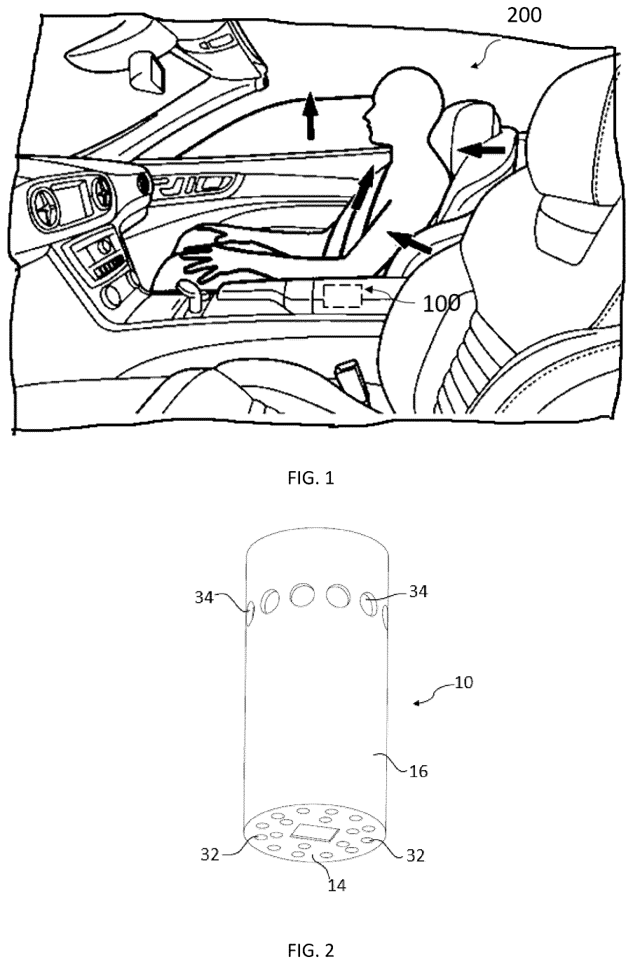

FIG. 1 generally illustrates a schematic diagram of a motor vehicle 200 with a fragrance unit 10 according to one or more embodiments of the present disclosure. It should be understood that in the context of the present disclosure, the vehicle 200 implementing the present disclosure may refer to any means of transportation, including but not limited to fossil fuel vehicles, electric vehicles such as plug-in hybrid electric vehicles (PHEVs), full hybrid electric vehicles (FHEVs), mild hybrid electric vehicles (MHEVs), or battery electric vehicles (BEVs), and so on. The vehicles may include mobility related components such as engine, electric motor, transmission, suspension, driveshaft, and/or wheels. The vehicles can be non-autonomous, semi-autonomous (for example, some conventional motion functions are autonomously controlled by the vehicle), or autonomous (for example, motion functions are autonomously controlled by the vehicle without direct user input).

Referring to FIGS. 2 to 5, one aspect of the present disclosure provides a fragrance unit 10, including a fragrance box 12, a fragrance medium 20 located inside the fragrance box 12, and a fluid channel 30 extending along a first direction of the fragrance box 12 and having an inlet 32 and an outlet 34. The fluid channel 30 is configured to allow airflow to flow into the fluid channel 30 from the inlet 32, pass through the fragrance medium 20, and then exit the fluid channel 30 from the outlet 34. Wherein the fluid channel 30 is arranged around the fragrance medium 20. The fragrance box 12 includes a bottom wall 14 and a side wall 16; with one of the inlet 32 and an outlet 34 located on the bottom wall 14 and the other on the side wall 16. The fluid channel 30 may include a channel through which the airflow flows from the inlet 32 to the outlet 34.

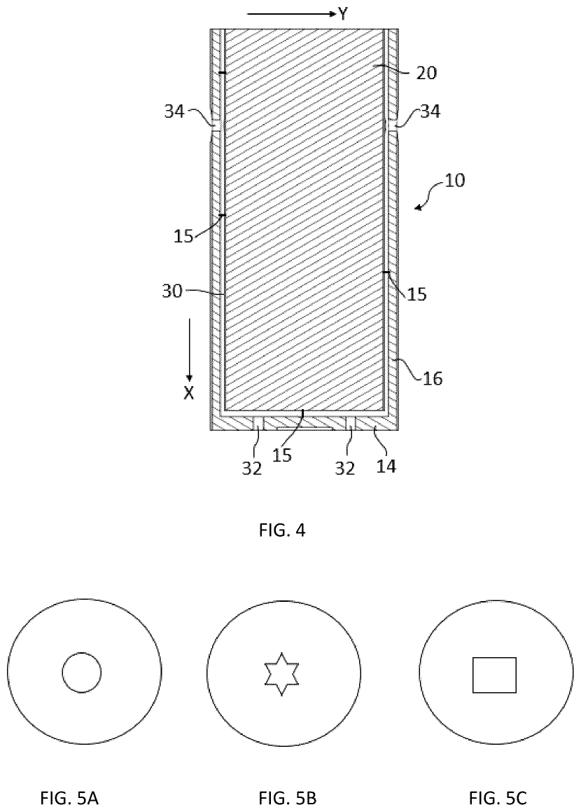

In one embodiment of the present disclosure, the first direction X may be the direction where the longest edge of the fragrance box 12 is located. As shown in FIG. 4, the first direction X can be a length direction of the fragrance box 12. In another embodiment, the fragrance box 12 is ellipsoid, and the first direction X may be the direction where the longest curvature of the ellipsoid is located, thus the fluid channel 30 can be maximized in a limited space, and the airflow through the channel is larger, making it possible for the fragrance entering the cabin to be more uniform.

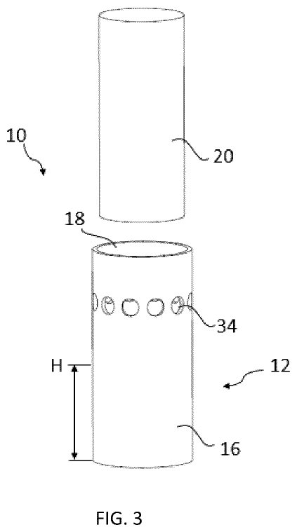

The fragrance box 12 can have various shapes and has a bottom wall 14 and a side wall 16. In one embodiment, as shown in FIG. 2, the fragrance box 12 may be cylindrical, including the bottom wall 14 and the side wall 16 connected to the bottom wall 14. In one embodiment, as shown in FIG. 4, the side wall 16 can extend along the first direction X from the edge of the bottom wall 14; in another embodiment, as shown in FIG. 3, the bottom wall 14 can also be located at a middle section H of the side wall 16. In another embodiment, the fragrance box 12 may also be ellipsoid, the bottom wall 14 may extend inward from an end of the ellipsoid to approximately one-fifth of its overall length, and the side wall 16 may include the middle section of the ellipsoid. In yet another embodiment, the fragrance box 12 can also be in other shapes such as rectangular, conical, or polyhedral.

In one embodiment of the present disclosure, as shown in FIGS. 2 to 4, the inlet 32 is provided on the bottom wall 14, and the outlet 34 is provided on the side wall 16 and located at one end of the side wall 16 away from the bottom wall 14. In another embodiment, the inlet 32 may be provided at one end of the side wall 16 away from the bottom wall 14, and the outlet 34 may be provided on the bottom wall 14. This can make the distance between the inlet 32 and the outlet 34 larger, resulting in a longer length of the fluid channel 30 and a longer flow time through the fragrance medium 20. This not only enhances the utilization rate of the fragrance medium 20 and prolongs the usage time of the fragrance, but also makes the fragrance more uniform.

Furthermore, as shown in FIG. 3, the fragrance box 12 has a first opening 18 opposite to the bottom wall 14, through which the fragrance medium 20 is taken. The inlet 32 or outlet 34 is located on the side wall 16, spaced apart and adjacent to the first opening 18. The inlet 32 or outlet 34 is arranged around the side wall 16, wherein, the term “adjacent to” can be understood as the inlet 32 or the first opening 18 being located along the first direction X at one end of the side wall 16 closer to the first opening 18. In one embodiment, there are multiple inlets 32 or outlets 34, which are evenly spaced around the side wall 16. In another embodiment, there is one inlet 32 or outlet 34, which is arranged around the side wall 16.

The first opening 18 has a size larger than the size of the fragrance medium 20 in a second direction Y, wherein the second direction Y is perpendicular to the first direction X. This facilitates the retrieval of the fragrance medium through the first opening 18, improving the convenience of operation, and creates a gap between the fragrance box 12 and the fragrance medium 20, providing the possibility of forming a uniform airflow channel.

The fragrance medium 20 has a projection on the bottom wall 14. In one or more embodiments, the inlet 32 or outlet 34 is provided within the projection of the fragrance medium 20 on the bottom wall 14, and the projection of the fragrance medium 20 on the bottom wall 14 covers the inlet 32 or outlet 34. In one embodiment, there are multiple inlets 32 or outlets 34, which are evenly spaced around the projection of the fragrance medium 20 on the bottom wall 14. Therefore, the airflow can flow through the fragrance medium 20 in multiple different directions, bringing the fragrance emitted by the fragrance medium 20 into the vehicle from multiple directions, better ensuring that the fragrance is evenly distributed at different positions of the fragrance medium 20, reducing the possibility of residual fragrance medium 20 at a certain angle when replacing the fragrance medium 20, and further improving the utilization rate of the fragrance medium 20. In another embodiment, the inlet 32 or outlet 34 may be provided only one inlet or one outlet, located at the center of the projection of the fragrance medium 20 on the bottom wall 14.

In some embodiments, the inlet 32 and outlet 34 may be holes of various shapes. When the inlet 32 or outlet 34 are provided only one, the hole may include various variations such as circular, star-shaped, rectangular-shaped (e.g., square-shaped), cross-shaped or elliptical as shown in FIGS. 5A to 5E, respectively. The hole may include other variations such as diamond-shaped, inclined, or ellipsoid. Technicians in this field can provide other possible variations according to actual design needs. When the hole is star- or cross-shaped, not only can it save materials, but also can it make the airflow flow through multiple angles, resulting in more uniform fragrance and enhancing user experience and satisfaction.

During the storage or transportation of the fragrance unit 10, the first opening 18, the inlet 32, and the outlet 34 are covered with seals, and the seals 18 can seal the fragrance unit 10 when the fragrance is not in use. In one embodiment, the seal 18 may be formed from a removable film to prevent fragrance leakage during the storage or transportation. When using the fragrance unit 10, the film can be removed and the fragrance unit 10 can be installed on the corresponding component.

The fragrance medium 20 can take different forms, such as solid or liquid. In one embodiment, as shown in FIG. 3, the fragrance medium 20 can be solid that is fixed inside the fragrance box 12 through a bracket 15 located inside the fragrance box 12 and defines the fluid channel 30 with the fragrance box 12. The fluid channel 30 is located between the fragrance box 12 and the fragrance medium 20. In another embodiment, the fluid channel 30 may also be an independently configured channel. The bracket can be arranged around a perimeter of the fragrance medium and between the fragrance medium and the bottom wall. Technicians in this field can make corresponding changes to the position and quantity of the brackets according to design needs. These changes are included within the scope of protection of the present disclosure and other substitutions, modifications, or omissions may be made to the design and settings of the above embodiments and other exemplary embodiments without departing from the spirit of the present disclosure.

The fragrance medium 20 can have various shapes that match the fragrance box 12, such as cylindrical, cuboid, or conical, for example. Correspondingly, the projection of the fragrance medium 20 on the bottom wall 14 has a shape that matches the shape of fragrance medium 20, such as circular, rectangular, or polygonal.

As shown in FIGS. 2 to 4, in this illustrative embodiment, there are multiple inlets 32 and outlets 34, respectively. Wherein the multiple inlets 32 are evenly spaced within the projection of the fragrance medium 20 on the bottom wall 14. The outlets 34 are located on the side wall 16 and adjacent to the first opening 18. The multiple outlets 34 are evenly spaced around the side wall 16. Multiple fluid channels 30 are formed between the inlets 32 and the outlets 34 through which the gas flows. This allows airflow to flow into different fluid channels 30 from the multiple inlets 32, and then flow through different directions and angles of the fragrance medium 20 after entering the fluid channels 30, and then flow out of the fluid channels 30 through the multiple outlets 34. This can allow airflow to pass through different positions and angles of the fragrance medium 20, making the overall fragrance of the fragrance medium 20 more uniform. This is more conducive to introducing the fragrance emitted by the fragrance medium 20 into the cabin from different directions, not only enhancing the utilization rate of the fragrance medium 20 and extending its usage time, but also making the fragrance more uniform. The performance of the fragrance unit 10 has been improved, which is conducive to enhancing user experience and satisfaction.

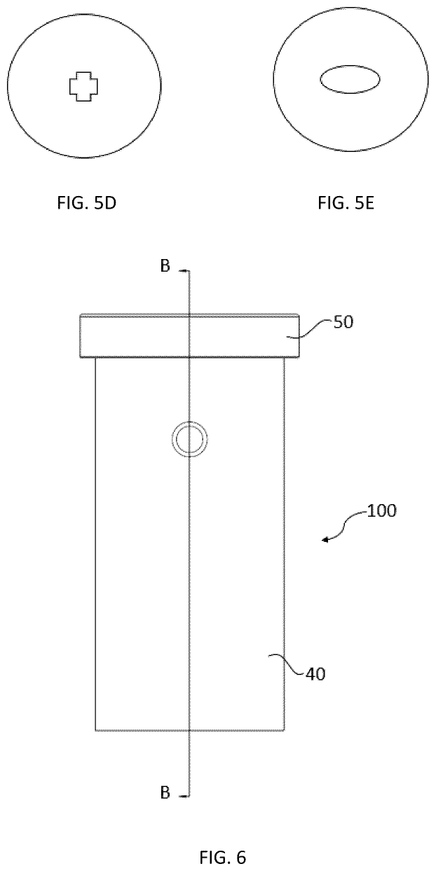

As shown in FIGS. 6 to 9, according to another aspect of the present disclosure, a vehicle fragrance assembly 100 is also provided. FIG. 6 shows an overall schematic diagram of the vehicle fragrance assembly 100. The vehicle fragrance assembly 100 may include the fragrance unit 10 described in any of the above embodiments. It should be understood that all the embodiments, features, and advantages described above for the fragrance unit 10 according to the first aspect of the present disclosure are equally applicable to the in vehicle fragrance assembly 100 according to the other aspect of the present disclosure, without conflicting with each other. That is to say, all the embodiments described above and their variations can be directly applied and combined here. For the sake of simplicity in this disclosure, it will not be repeated here.

The vehicle fragrance assembly 100 may also include a carrier 40, an accommodation cavity 42 is formed inside the carrier 40, and the fragrance unit 10 is located inside the accommodation cavity 42. The carrier 40 is formed by an external shell.

As shown in FIG. 1, the fragrance assembly 100 can be integrated in different positions of the vehicle 200. In one embodiment, the vehicle fragrance assembly 100 can be installed on a central armrest or backstage armrest for user control to turn on or off the fragrance assembly. In another embodiment, the fragrance assembly 100 can also be installed on a dashboard. In another embodiment, the vehicle fragrance assembly 100 can also be installed on a roof console to enhance the user perception of fragrance and improves the user experience.

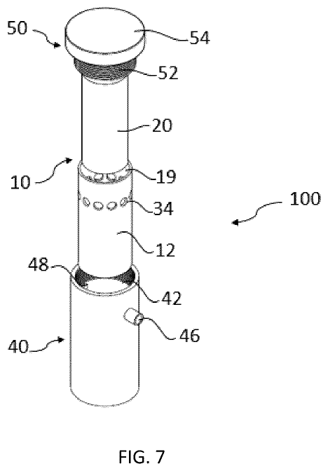

As shown in FIG. 7, the carrier 40 has an upward facing second opening 48, through which the fragrance unit 10 is inserted into or removed from the accommodation cavity 42.

The vehicle fragrance assembly 100 may also include a cover 50 for closing the second opening 48. The cover 50 includes a main body 54 and a connecting portion 52 extending from the main body 54 towards the accommodation cavity 42. The main body 54 can cover the second opening 48. The connecting portion 52 is connected to the carrier 40 on one side in a sealed manner, and to the fragrance box 12 on the other side.

Specifically, as shown in FIG. 9, the connecting portion 52 has a first side 56 facing the accommodation cavity 42 and a second side 58 away from the accommodation cavity 42. The first side 56 and the second side 58 are arranged opposite each other. In one embodiment, the first side 56 is threaded to the fragrance box 12, and the second side 58 is threaded to the carrier 40. In another embodiment, the first side 56 can also be snap connected to the fragrance box 12, and the second side 58 can also be snap connected to the carrier 40.

Thus, the cover 50 can be detachably connected to the fragrance unit and the carrier. During use, if the fragrance is consumed, only the fragrance unit 10 needs to be replaced, and the cover 50 can be reused, making it more environmentally friendly. The cover 50 can have various personalized designs according to user needs, enhancing the overall aesthetics of the cover 50 and the fragrance assembly 100. The cover 50 can be exposed inside the vehicle, and different materials can be selected according to user needs, such as ceramic, crystal, wood, glass, stone and other decorative materials, providing the user with more choices and enhancing user satisfaction.

As shown in FIG. 9, a seal 18 is installed below the main body, and the seal 18 is arranged around the connecting portion 52. This can seal the cover 50 and the carrier 40 around the second opening 18, reducing the possibility of airflow flowing out between the cover 50 and the carrier 40, allowing the airflow to flow out along the fluid channel 30.

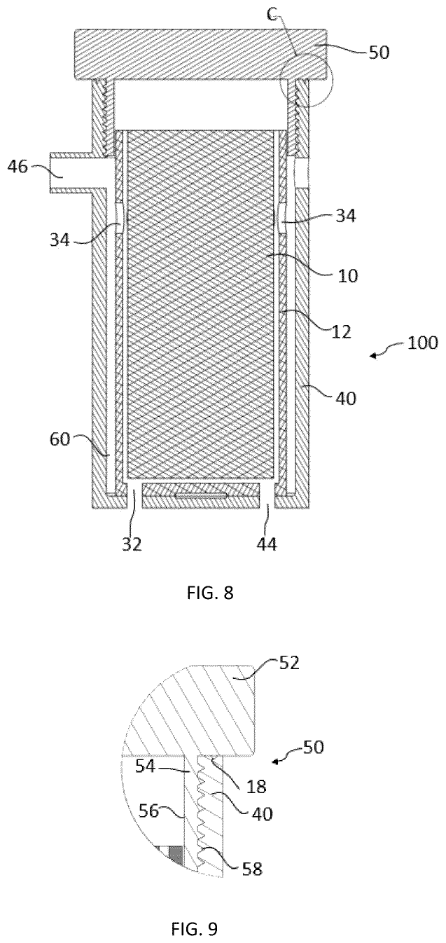

As shown in FIG. 8, the carrier 40 is also provided with an inlet hole 44 and an outlet hole 46. The airflow enters from the inlet hole 44, then flows into the fluid channel 30 through the inlet 32, then passes through the fragrance medium 20, then flows out from the outlet 34, then exits the fluid channel 30 through the outlet hole 46 and then enters the interior of the vehicle.

The inlet hole 44 is located adjacent to the inlet 32, and the outlet hole 46 is located adjacent to the outlet 34. As shown in FIG. 6, in one embodiment, the inlet hole 44 is aligned with the inlet 32, and the outlet hole is aligned with the outlet 34. In another embodiment, the outlet hole is located downstream of the inlet 32 along the flow direction of the airflow.

As shown in FIG. 8, there is a gap 60 between the fragrance box 12 and the carrier 40, and the gap 60 is in fluid communication with the fluid channel 30. Both the gap 60 and the fluid channel 30 extend along the first direction X. The bottom wall 14 of the fragrance box 12 is in contact with a bottom end of the carrier 40. When the vehicle fragrance assembly 100 is not in use, the inlet hole 44 and/or outlet hole 46 are closed, preventing the formation of airflow. The fragrance naturally evaporated from the fragrance medium 20 can be retained in the gap. When the vehicle fragrance assembly 100 is turned on, the fragrance retained in the gap can flow out of the outlet 46 into the vehicle in a short period of time, which can enhance fragrance efficiency, reduce response time, and further enhance user experience and satisfaction.

When the fragrance medium 20 has evaporated, the fragrance unit 10 can be replaced. The user can open the cover 50, take out the used fragrance unit 10 from the second opening 48, and then insert a new fragrance unit 10 for continued use. The cover 50 can be reused.

The present disclosure summarizes aspects of the embodiments and should not be used to limit the claims. Other implementations are contemplated in accordance with the techniques described herein, as will be apparent upon examination of the following drawings and detailed description, and such implementations are intended to be within the scope of this disclosure.

The present disclosure provides for an enhanced fragrance unit and a fragrance assembly located inside a vehicle. An aspect of the embodiment of the present disclosure relates to a fragrance unit. The fragrance unit includes a fragrance box, a fragrance medium located inside the fragrance box, and a fluid channel extending along a first direction of the fragrance box and having an inlet and an outlet, the fluid channel being configured to allow airflow to flow into the fluid channel from the inlet, pass through the fragrance medium, and then exit the fluid channel from the outlet, wherein the fluid channel is arranged around the fragrance medium; and wherein the fragrance box comprises a bottom wall and a side wall, with one of the inlet and the outlet located on the bottom wall and the other on the side wall.

According to an embodiment of the present disclosure, the fragrance medium is solid that is fixed to the fragrance box through a bracket located inside the fragrance box and defines the fluid channel with the fragrance box.

According to an embodiment of the present disclosure, multiple inlets or multiple outlets are provided, wherein the multiple inlets and/or the multiple outlets are evenly spaced around a projection of the fragrance medium on the bottom wall.

According to an embodiment of the present disclosure, multiple inlets or multiple outlets are provided, and a projection of the fragrance medium on the bottom wall covers the multiple inlets or the multiple outlets.

According to an embodiment of the present disclosure, the fragrance box has a first opening opposite to the bottom wall, and the inlet or outlet is located on the side wall, spaced apart from and adjacent to the first opening.

According to an embodiment of the present disclosure, multiple inlets or multiple outlets are provided, and the multiple inlets or the multiple outlets are evenly arranged around the side wall.

According to an embodiment of the present disclosure, one inlet or one outlet is provided, and the inlet or the outlet is cross-shaped, star-shaped, circular or square-shaped.

According to an embodiment of the present disclosure, the fragrance box has an opening opposite to the bottom wall and having a size larger than a radial size of the fragrance medium.

Another aspect of the present disclosure also provides a fragrance assembly located inside a vehicle, including a fragrance unit comprising a fragrance box and a fragrance medium located inside the fragrance box, a carrier with an accommodation cavity formed internally, within which the fragrance unit is located, and a cover comprising a connecting portion that is connected to the carrier on one side in a sealed manner and to the fragrance box on the other side.

According to an embodiment of the present disclosure, a gap in fluid communication with to the fluid channel is formed between the fragrance box and the carrier.

According to an embodiment of the present disclosure, the gap and the fluid channel extend along the first direction.

The above-mentioned embodiments are possible examples of implementations of the present disclosure and are given only for the purpose of enabling those skilled in the art to clearly understand the principles of the invention. It should be understood by those skilled in the art that the above discussion to any embodiment is only illustrative, and is not intended to imply that the disclosed scope of the embodiments of the present disclosure (including claims) is limited to these examples. Under the overall concept of the invention, the technical features in the above embodiments or different embodiments can be combined with each other to produce many other their variants in different aspects of embodiments of the invention that is not provided in detailed description for the sake of brevity. Therefore, any omission, modification, equivalent replacement, improvement, etc. made within the spirit and principle of the embodiment of the invention shall be included in the scope of protection claimed by the invention.

It is to be understood that variations and modifications can be made on the aforementioned structure without departing from the concepts of the present disclosure, and further it is to be understood that such concepts are intended to be covered by the following claims unless these claims by their language expressly state otherwise.

What is claimed is:

1. A fragrance unit, comprising:
a fragrance box having a bottom wall and a side wall;
a fragrance medium located inside the fragrance box; and
a fluid channel extending along a first direction of the fragrance box and having an inlet and an outlet with one of the inlet and the outlet located on the bottom wall and the other on the side wall, the fluid channel being configured to allow airflow to flow into the fluid channel from the inlet, pass through the fragrance medium, and then exit the fluid channel from the outlet, wherein the fluid channel is arranged around the fragrance medium.

2. The fragrance unit as claimed in claim 1, wherein the fragrance medium is solid that is fixed to the fragrance box through a bracket located inside the fragrance box and defines the fluid channel with the fragrance box.

3. The fragrance unit as claimed in claim 1, wherein multiple inlets or multiple outlets are provided.

4. The fragrance unit as claimed in claim 3, wherein the multiple inlets or the multiple outlets are evenly spaced around a projection of the fragrance medium on the bottom wall.

5. The fragrance unit as claimed in claim 3, wherein a projection of the fragrance medium on the bottom wall covers the multiple inlets or the multiple outlets.

6. The fragrance unit as claimed in claim 1, wherein the fragrance box has a first opening opposite to the bottom wall, and the inlet or outlet is located on the side wall, spaced apart from and adjacent to the first opening.

7. The fragrance unit as claimed in claim 6, wherein multiple inlets or multiple outlets are provided, and the multiple inlets or the multiple outlets are evenly arranged around the side wall.

8. The fragrance unit as claimed in claim 1, wherein one inlet or one outlet is provided, and the one inlet or the one outlet is circular, star-shaped, rectangular-shaped, cross-shaped or elliptical.

9. The fragrance unit as claimed in claim 1, wherein one inlet or one outlet is provided, and the one inlet or the one outlet is cross-shaped, circular or square-shaped.

10. The fragrance unit as claimed in claim 1, wherein the fragrance unit is configured to be located on a fragrance assembly inside a vehicle.

11. A fragrance assembly located inside a vehicle, the fragrance assembly comprising:
a fragrance unit comprising a fragrance box, a fragrance medium located inside the fragrance box, and a fluid channel extending along a first direction of the fragrance box and arranged around the fragrance medium, the fluid channel having an inlet and an outlet;
a carrier with an accommodation cavity formed internally, within which the fragrance unit is located; and
a cover comprising a connecting portion that is connected to the carrier on one side in a sealed manner and to the fragrance box on another side.

12. The fragrance assembly as claimed in claim 11, wherein a gap in fluid communication with the fluid channel is formed between the fragrance box and the carrier.

13. The fragrance assembly as claimed in claim 12, wherein the gap and the fluid channel extend along the first direction.

14. The fragrance assembly as claimed in claim 11, wherein the fragrance medium is solid that is fixed to the fragrance box through a bracket located inside the fragrance box and defines the fluid channel with the fragrance box.

15. The fragrance assembly as claimed in claim 11, wherein multiple inlets or multiple outlets are provided.

16. The fragrance assembly as claimed in claim 11, wherein the fragrance box has a first opening opposite to the bottom wall, and the inlet or outlet is located on the side wall, spaced apart from and adjacent to the first opening.

17. The fragrance assembly as claimed in claim 11, wherein one inlet or one outlet is provided, and the one inlet or the one outlet is circular, star-shaped, rectangular-shaped, cross-shaped or elliptical.

18. A vehicle comprising:
a fragrance assembly located inside a vehicle, the fragrance assembly comprising:
a fragrance unit comprising a fragrance box, a fragrance medium located inside the fragrance box, and a fluid channel extending along a first direction of the fragrance box and arranged around the fragrance medium, the fluid channel having an inlet and an outlet;
a carrier with an accommodation cavity formed internally, within which the fragrance unit is located; and
a cover comprising a connecting portion that is connected to the carrier on one side in a sealed manner and to the fragrance box on another side.

19. The vehicle as claimed in claim 18, wherein the fragrance assembly has a gap in fluid communication with the fluid channel formed between the fragrance box and the carrier.

20. The vehicle as claimed in claim 19, wherein the gap and the fluid channel extend along the first direction.
